(19) United States
(12) Patent Application Publication
(10) Pub. No.: US 2013/0059379 A1
(43) Pub. Date: Mar. 7, 2013

(54) METHOD FOR THE GENERATION OF A CIK CELL AND NK CELL POPULATION

(71) Applicant: Ingo Schmidt-Wolf, Bonn (DE)

(72) Inventor: Ingo Schmidt-Wolf, Bonn (DE)

(21) Appl. No.: 13/580,980

(22) PCT Filed: Feb. 24, 2010
(86) PCT No.: PCT/EP2010/001148

§ 371 Date: Nov. 12, 2012

(51) Int. Cl. C12N 5/0783

(52) U.S. Cl. 435/372.3; 435/377; 435/325

(57) ABSTRACT

The present invention is directed a method for the generation and/or expansion of CIK cell and/or NK cells by culturing peripheral blood cells in the presents of cytokines. The cytokines used in the method comprise interleukin 15 (IL-15) and interleukin 7 (IL-7), possibly in combination with further cytokines like interleukin 2 (IL-2) stem cell factor (SCF) and Fms-related tyrosine kinase 3 ligand (FLT3 ligand).

The present invention is directed a method for the generation and/or expansion of CIK cell and/or NK cells by culturing peripheral blood cells in the presents of cytokines. The cytokines used in the method comprise interleukin 15 (IL-15) and interleukin 7 (IL-7), possibly in combination with further cytokines like interleukin 2 (IL-2), stem cell factor (SCF), and Fms-related tyrosine kinase 3 ligand (FLT3 ligand). The present invention is also directed to a kit for the generation and/or expansion of CIK cells and/or NK cells comprising IL-15 and IL-7, possibly in combination with further cytokines like IL-2, SCF, and/or FLT3 LIGAND. Further the present invention is directed to a CIK and/or NK cell population obtained by such methods.

### BACKGROUND

In the past decade, a protocol has been firmly established to rapidly and reproducibly expand in vitro T cells with NK phenotype termed cytokine-induced killer (CIK) cells [1]. These cells are characterized by a very high cytolytic potential, starting from human blood from normal donors or from leukemia/lymphoma patients [2,3]. It is considered that the cytotoxicities of CIK cells and another potent immune cytolytic effector, natural killer (NK) cells, both show to be neither MHC restricted nor mediated by the T-cell receptor but via perforin mediated mechanisms [1,4,5]. Infusion of activated CIK cells can promote graft versus leukemia (GVL) or anti-tumor effect without severe transfusion-related graft versus host disease (GVHD) [3, 6]. Some studies demonstrated that CIK cells have stronger antitumor activity as compared to NK and LAK cells and almost no cytotoxicity on normal hematopoiesis progenitor cells [7].

Many clinical outcomes of hemotopoietic stem cell transplantations (HSCT) clued up with a relationship between GVHD and GVL, and it is now clear that the GVL effect is responsible for impeding leukemia relapse after HSCT or chemotherapy. Cord blood transplantation is performed since 20 years. Though the incidence and the severity of acute/chronic GVHD in cord blood transplantation (CBT) are less than that of bone marrow transplantation (BMT) [8,9,10], these are not implying that after CBT, the relapse rate, disease-free survival and overall survival of children with acute leukemia are less than that of BMT [11]. Presence of immature killer progenitor cells in cord blood promise the potential GVL effect following CBT [12].

CIK and NK cells are both important antitumor effectors in the immunotherapy of malignancies, at the present time, the extensive used cultural system of expanding CD3+CD56+CIK cells has almost no effect on expanding CD3−CD56+NK cells, but with a high ratio of CD3+CD8+T cells [13,14]. Excessive T cells inside of the CIK cells culture system are of no concern eliminating tumor cells for lacking prior tumor-specific antigen stimulating but increasing the risk of cell-therapeutic related GVHD [15,16]. Though these unwanted CD8+ T cells can be removed by a magnetic separating device, such as Clinimax system from Miltenyi, the high cost and cumbersome to operate hindering its widely clinical application. So, setting up a cultural system in which possibly expanding CIK/NK cells synchronously in a same culture system would be valuable, economical and practical for clinical application of CIK/NK cells.

Accordingly, it was an object underlying the present invention to provide a new method for the generation and/or expansion of CIK cells and/or NK cells which avoids the disadvantages of the previously used methods and allows the generation and/or expansion of CIK cells and/or NK cells with high expanding multiples. It was a further object of the present invention to provide a kid for the generation and/or expansion of CIK cells and/or NK cells and to provide a population of CIK cells and/or NK cells generated by the method.

The above mentioned problem is solved by a method for generating and/or expansion of CIK cells and/or NK cells, the method comprising culturing blood cells in the presence of a culture medium comprising interleukin 15 (IL-15) and interleukin 7 (IL-7).

The present inventor has surprisingly found, that the combination of the cytokins IL-15 and IL-7 allows a very effective method for generating and/or expansion of CIK cells and/or NK cells which gives better results than the previously known methods.

In a preferred embodiment, the culture medium used in the method additionally comprises interleukin 2 (IL-2).

In a further preferred embodiment, the culture medium used in the method according to the present invention further includes stem cell factor (SCF).

In a further preferred embodiment, the culture medium used in the method according to the present invention additionally comprises Fms-related tyrosine kinase 3 ligand (FLT3 ligand).

FLT3 ligand is a naturally occuring glycoprotein which is a hematopoietic four helical bundle cytokine. In previous studies FLT3 ligand has been found to simulate the proliferation and differentiation of various blood cell progenitors.

In a particularly preferred embodiment of the present invention the cell culture medium used for the method comprises one of the following combination of cytokines: IL-15, IL-7, and IL-2; IL-15, IL-7, IL-2, and SCF; IL-15, IL-7, IL-2 and FLT3 ligand; IL-15, IL-7, IL-2, SCF and FLT3 ligand.

The present inventivor has found that a wide range of concentrations of the respective cytokines can be used in the method of the present invention. Particularly preferred embodiments employ the cytokines in the following amounts:

In a preferred embodiment, the culture medium used in the method according to the present invention comprises IL-15 in an amount of about 4 to 400 ng/ml, more preferably in an amount of about 10 to 100 ng/ml, and even more preferably in an amount 20 to 60 ng/ml. The present inventors have obtained particularly good results when the culture medium comprised IL-15 in an amount of about 40 ng/ml.

In a preferred embodiment, the culture medium used in the method according to the present invention comprises IL-7 in an amount of about 4 to 400 ng/ml, more preferably in an amount of about 10 to 100 ng/ml, and even more preferably in an amount 20 to 60 ng/ml. The present inventors have obtained particularly good results when the culture medium comprised IL-7 in an amount of about 40 ng/ml.

In a preferred embodiment, the culture medium used in the method according to the present invention comprises IL-2 in an amount of about 8 to 800 ng/ml, more preferably in an amount of about 20 to 200 ng/ml, and even more preferably in an amount 40 to 120 ng/ml. The present inventors have obtained particularly good results when the culture medium comprised IL-2 in an amount of about 80 ng/ml.

In a preferred embodiment, the culture medium used in the method according to the present invention comprises SCF in an amount of about 4 to 400 ng/ml, more preferably in an amount of about 10 to 100 ng/ml, and even more preferably in an amount 20 to 60 ng/ml. The present inventors have obtained particularly good results when the culture medium comprised SCF in an amount of about 40 ng/ml.

In a preferred embodiment, the culture medium used in the method according to the present invention comprises FLT3 ligand in an amount of about 4 to 400 ng/ml, more preferably in an amount of about 10 to 100 ng/ml, and even more preferably in an amount 20 to 60 ng/ml. The present inventors have obtained particularly good results when the culture medium comprised FLT3 ligand in an amount of about 40 ng/ml.

In a preferred embodiment of the method according to the present invention, the culture medium used in the method of according to the present invention comprises IL-15 in an amount of 4 to 400 ng/ml and IL-7 in an amount of 4 to 400 ng/ml.

In another preferred embodiment of the method according to the present invention, the culture medium according to the present invention comprises IL-15 in an amount of about 4 to 400 ng/ml, IL-7 in an amount of about 4 to 400 ng/ml and IL-2 in an amount of about 8 to 800 ng/ml.

In another preferred embodiment of the method according to the present invention, the culture medium comprise IL-15 in an amount of about 4 to 400 ng/ml, IL-7 in an amount of about 4 to 400 ng/ml, IL-2 in an amount of about 8 to 800 ng/ml and SCF in an amount of about 4 to 400 ng/ml.

In another preferred embodiment of the method according to the present invention, the culture medium comprises IL-15 in an amount of 4 to 400 ng/ml, IL-7 in an amount of 4 to 400 ng/ml, IL-2 in an amount of 8 to 800 ng/ml and FLT3 ligand in an amount of 4 to 400 ng/ml.

In another preferred embodiment of the method according to the present invention, the culture medium comprise IL-15 in an amount of about 4 to 400 ng/ml, IL-7 in an amount of about 4 to 400 ng/ml, IL-2 in an amount of about 8 to 800 ng/ml, SCF in an amount of about 4 to 400 ng/ml and FLT3 ligand in an amount of about 4 to 400 ng/ml.

In a preferred embodiment of the method according to the present invention, the blood cells used are peripheral blood cells.

In the context of the present invention the term peripheral blood cells is to be understood to be the cellular components of blood, consisting of red blood cells, white blood cells, and platelets, which are found within the circulating pool of blood and not sequestered within the lymphatic system, spleen, liver, or bone marrow.

In a particularly preferred embodiment, the method according to the present invention comprises culturing of cord blood cells.

In the context of the present invention, the term cord blood is to be understood to refer to the blood which remains in the placenta and in the attached umbilical cord after child birth.

In preferred embodiment, the method according to the present invention comprises the culturing of mammal blood cells.

In another preferred embodiment, the method according to the present invention comprises the culturing of human blood cells.

In particular a preferred embodiment, the method according to the present invention comprises the culturing of human blood cells derived from cord blood.

The problem underlying the present invention is further solved by a kit for the generation and/or expansion of CIK cells and/or NK cells comprising IL-15, IL-7, and IL-2; and facultatively either
SCF, or
FLT3 ligand, or
SCF and FLT3 ligand.

In a preferred embodiment, the kid according to the present invention comprises IL-15, IL-7, and IL-2.

In another preferred embodiment, the kid according to the present invention comprises IL-15, IL-7, IL-2, and SCF.

In another preferred embodiment, the kid according to the present invention comprises IL-15, IL-7, IL-2 and FLT3 ligand.

In another preferred embodiment, the kid according to the present invention comprises IL-15, IL-7, IL-2, SCF, and FLT3 ligand.

The present invention is further directed to a population of CIK cells and/or NK cells obtained by a method as described above or by use of a kid as described above.

Our research has shown that with the stimulating of effective NK cells growth cytokines, such as the combination of IL-7 and IL-15, preferably in combination with IL-2, blood derived CIK and NK cells, in particular cord blood derived CIK and NK cells can both be expanded simultaneously. In this study we further found that cytokines such as stem cell factor (SCF) and FLT3 ligand, which are acting on the early period of hematopoiesis stem cells in cord blood, combined with IL-2, IL-7 and IL-15, will be efficient for inducing and expanding cord blood derived CIK and NK cells. So we explored the possibilities of establishing a highly effective modified expanding system for CB-CIK/NK cells and compared the cytotoxicity of these CIK/NK cells from various cultural protocols against the K562 cell line by XTT assay.

Material and Methods

Generation of CB-CIK/NK Cells by Different Protocols

Umbilical cord blood mononuclear cells (MNC) were isolated by Ficoll density gradient (1.077±0.002 g/ml, Jinmei, Shenzhen, GD, China) centrifugation, washed, and resuspended at 1×10⁶ cells/ml in Iscove's Modified Dulbecco's Medium (IMDM, Gibco, Grand Island, N.Y., USA) containing 10% fetal calf serum (FCS, Sigma, St. Louis, Mo., USA), penicillin 100 U/mL, streptomycin 100 mg/mL, 2 mmol/L L-glutamine, and 50 μmol/L 2-mercaptoethanol. According to the different combination of the cytokines, there were three experimental groups. The protocol for expanding CB-CIK/NK cells in group A was SCF (Becton Dickinson, San Jose, Calif., USA, 40 ng/ml) , IL-2 (Becton Dickinson, San Jose, Calif., USA, 80 ng/ml), IL-7 (Becton Dickinson, San Jose, Calif., USA, 40 ng/ml) and IL-15 (Becton Dickinson, San Jose, Calif., USA, 40 ng/ml). The protocol of group B was mainly the same as that of group A but with FLT3 ligand (Becton Dickinson, San Jose, Calif., USA, 40 ng/ml) additionally. The protocol of group C was IL-2, IL-7 plus IL-15. Various cytokines were added into different groups on day 1. Cells were incubated at 37° C. in a humidified atmosphere of 5% CO₂, fed every three days in fresh complete IMDM and various types of cytokines.

Triplication experiments were designed for the same donor, and there were fifteen different CB donors tested. Representative results shown here were from nine separate experiments of three different donors.

Proliferation and Phenotype Analysis

On days 0, 14 and 21, viable CB-CIK/NK cells densities were determined by cell numbers calculating with a hemocytometer, and the phenotypes of the cells from various groups were identified by flow cytometry, respectively. Flow cytometry analysis CIK cells, NK cells, CD4+ T cells and CD8+ T cells were labeled with CD3-PerCP/CD56-PE, CD3-PerCP/CD4-FITC and CD3-PerCP/CD8-FITC (Becton Dickinson, USA). 5×10⁵ cells were resuspended in 20 μL of 2% newborn calf serum and 1% sodium azide in phosphate-buffered saline (PBS), and incubated with 10 μL of appropriate MoAbs for 30 min at 4° C. After incubation, the cells were washed twice and resuspended in 1.0 mL of assay buffer. Nonspecific binding was determined using irrelevant mouse immunoglobulin isotypes IgG1-FITC, IgG1-PE, IgG1-PerCP and IgG2b-PE. The fluorescence was analyzed by a Coulter FC500 flow cytometer (Beckman Coulter, Fullerton, Calif., USA).

The CB-CIK/NK cells were harvested on day 21, after being stained with 7-AAD and CD3-PerCP/CD56-PE of flow cytometry analysis showed that if the viable cells exceeded 95% and the percentage of CD3+CD56+ plus CD3−CD56+ cells above 60%, then the cells qualified for the next cytotoxic XTT assay as the effector cells.

Cell Lines

The human erythroleukemia cell line K562 was purchased from Cancer Institute of Sun Yat-sen University Cancer Center. The cells were maintained in RPMI 1640 supplemented with 10% FCS, 100 U/ml penicillin and 100 g/ml streptomycin and grown at 37° C. in a humidified atmosphere of 5% CO₂.

Cytotoxic Effects of CB-CIK/NK Cells Against K562 Cell Line by XTT Assay

The cytotoxic potential of the expanded CB-CIK/NK cells by different protocols against K562 cell line were measured by (2,3)-bis-(2-methoxy-4-nitro-5-sulfophenyl)-(2H) -tetrazolium-5-carboxanilide (XTT) (Sigma, St. Louis, Mo., USA) assay as described in previous studies [17], XTT was prepared in Dulbecco's PBS (PBS, Gibco, Grand Island, N.Y., USA) at 0.25 mg/mL, color developing reagent, (2,3)-dimethoxy-5-methyl-(1,4)-benzoquinone (coenzyme Q, Sigma, St. Louis, Mo., USA) was prepared at concentration of 0.05 mg/mL in PBS. Fresh XTT stock solutions prepared for each experiment contained 1 mL XTT with 8 uL coenzyme Q. 1×10⁴ cells/well target cells (K562 cells) were incubated in triplicate sets with effector cells (CB-CIK/NK cells) in a U-bottom, 96-well culture plates, with ratios of effector cells to target cells as 20:1 and 10:1. Both controls of effector and target cells were set up at the same time. After 4 hr incubation, all supernatants were collected and removed after centrifugation (3000 rpm for 10 min), a 150 uL volume of XTT stock solution containing coenzyme Q was then added and the plates were gently shaken in a shaker incubator. After an incubation period of 1 h at 37° C., the cells were pelleted by centrifugation(3000 rpm for 10 min) and 100 uL supernatants were transferred to a fresh 96-well plates, and the absorbance at 490 nm was measured using a microplate reader (Universal Microplate Reader, ELx 800 UV). Percent reduction in formazan produced was calculated using the following formula:

$$Cytotoxicity ( \% ) = 1 - \frac{A 490 ( effector + target ) - A 490 ( effector control )}{A 490 ( target control )} \times 100$$

Statistical Analysis

Data were presented as the mean±standard deviation. Statistical analysis was performed using a SPSS 16.0 software package. The quantitative data were compared using one-way analysis of variance and least significant difference (LSD) method. P values of less than 0.05 were considered statistically significant.

Results

Generation of Cytotoxic CB-CIK/NK Cells from Cord Blood

CD3+CD56+ CIK cells were rare (0.5%±0.2%) in uncultured CB, and CD3−CD56+ NK cells were 12.7%±6.4% on day 0. After stimulating of the combined SCF, FLT3 ligand, IL-2, IL-7 and IL-15, the proportion and multiples of the expanded CB-CIK/NK cells in group A, B and C observably higher than that of uncultured CB.

Compared with that of group C, after 21 days expansion, the percentage of CD3+CD56+ CIK cells in group B with five-cytokines peaked to 26.20%±4.05%, its significantly higher than that of group A (19.84%±2.11%). The proportion of CD3+CD56+ CIK cells in group B and C on day 21 were significantly higher than that of day 14, but to group A, no significant difference could be seen between these two time points. The phenotypic changes of proportion of CD3−CD56+ NK cells in various groups of our study were less obviously, in group A and B, that appeared to a plateau after two weeks in culture, and there were no significant differences in productive rate of CD3−CD56+ NK cells in group A and B between day 21 and 14. For group C, though on day 21,the proportion of CD3−CD56+ NK cells was significantly lower than that on day 14 and that of group A and B, that was about 21% (Table. 1, FIG. 1).

The absolute number of CB derived CD3+CD56+ CIK cells increased significantly after 14 to 21 days of culture, and on day 21, was expanded nearly 550 to 800-fold in various groups, and that of CD3−CD56+ NK cells was expanded nearly 15 to 48-fold in various groups with different protocol. According to group A and B, the expanded multiples of CIK and NK cells increased gradually with the extension of culture time, but to group C, that of NK cells was not to follow this tendency and with a drop on day 21 compared to day 14 (Table 2, FIG. 1).

Proportion of CD4+ T cells and CD8+ T Cells in CB-CIK/NK Cells Cultivation

There were only minor changes in the percentage of CD4+CD3+ T cells during CB-CIK/NK cells cultivation, and no significant difference could be seen between the ratio of CD4+CD3+ T cells on day 21 and day 14.The proportion of CD8+CD3+ T cells on day 21 decreased dramatically than that on day 14. When CB-CIK/NK cells harvested on day 21, the mixed-into CD8+CD3+ T cells were merely about 24%, 13% and 10% in group A, B and C, respectively (Table 3, FIG. 1).

Our result showed that even with only three cytokines, IL-2, IL-7 and Il-15, CD3+CD56+ CIK cells and CD3−CD56+ NK cells could be effective expanded simultaneously, and there were less CD8+CD3+ T cells in the cultivation, it was quiet different than those reported before, in which for CIK cells expanding, the protocols used almost had no effect on NK cells producing and there was a relatively high content of T cells inside.

Cytotoxicity of Expanded CB-CIK/NK Cells Against K562 Cell Line

The cytotoxic effect of CB-CIK/NK cells expanded in the presence of SCF, FLT3 ligand, IL-2, IL-7 and IL-15 were studied using K562 cell line as targets in a XTT cytotoxicity assay. All expanded CB-CIK/NK cells showed cytotoxic activity against the K562 cell line, and the cytotoxicity of effector:target ratio 20:1 was significantly higher than that of 10:1. The cytotoxic activity of group A was highest and significantly higher than that of other groups. There was no obvious difference in cytotoxicity against K562 cell line between group B and C (FIG. 2).

Discussion

CIK cells were shown to be a heterogeneous population with different cellular phenotypes that were generated by incubation of peripheral blood [1,18] or cord blood [19,20] mononuclear cells with various cytokines, such as anti-CD3 monoclonal antibody (m-Ab), IL-1, IL-2, IL-12 and interferon gamma. CD3+ T cells co-expressing the CD56 antigen were first described by Lanier [21] in 1986, and a remarkable expansion of this cellular subset has been obtained and developed by Schmidt-Wolf et al. [22]. The higher lytic activity against tumor cells of CIK cells was mainly due to the higher proliferation of CD3+CD56+ double positive cells, from the studies of Schmidt-Wolf [23] in the presence of anti-CD3 m-Ab, IL-1, IL-2 and interferon gamma after 14 days culturing, peripheral blood derived CD3+CD56+ double positive cells can increase of 754-fold. The application of anti-CD3 m-Ab and IL-1 were critical and optimal for the proliferation and cytotoxic activity of CIK cells [22], and this protocol has now been widely adopted as the “classical” protocol for expanding CIK cells.

The results from Lu [13] showed that by using the “classical” protocol, during the expansion course of peripheral blood derived CD3+CD56+ CIK cells and CD3−CD56+ NK cells on day 0, 10, 20 and 30, the ratio of CIK cells was 2.3%, 5.5%, 23% and 28%, respectively, and that of NK cells was only 12%, 5%, 3.9% and 2%, respectively. After one month, CD3+CD56+ cells expanded nearly 1000-fold, nevertheless, CD3−CD56+ NK cells expanded less than 10-fold under these culture conditions. Findings Ren [24] demonstrated that with the protocol mentioned above, CD56+ cells could increase from 8.8±0.3% to 43.1±4.2%, whereas the CD16+ cells with no change and sustained at about 8% during 15 days of culture. Another study [14] of peripheral blood derived CD3+CD56+ CIK cells expanded by these “classical” cytokines also demonstrated that after culturing the proportion of CD16+CD56+ cells decreased from 9.2±8.3% to 4.8±4.0%. The same protocol can also be used for cord blood derived CD3+CD56+ CIK cells expansion and leads to same low NK cells production [25]. All these studies indicate that the combination of anti-CD3 m-Ab, IL-1, IL-2 and interferon gamma used for CD3+CD56 CIK cells expansion had weakly expanding effect on CD3−CD56+ NK cells. For NK cells are also the important anti-tumor effectors in biotherapy [26, 27], it will be important for promoting of GVL effect after HSCT if we can induct and expand CIK and NK cells in one culture system simultaneously.

At present, CD3+CD56+ CIK cells were considered coming from CD3+CD56− T cells but not CD3−CD56+ NK cells [1, 13]. We have previously demonstrated that by using the combination of IL-2, IL-7 and IL-15 that successful expansion of both CD3+CD56+ CIK cells and CD3−CD56+NK cells from cord blood is possible. Here we explored the influence of two important early stage hematopoietic growth factors, SCF and FLT3 ligand together with the combination of IL-2, IL-7 and IL-15 on inducing and expanding of cord blood derived CIK and NK cells. It was verified that SCF in cooperation with IL-2 can stimulate cell proliferation and increase the sensitive of IL-2 receptors [28 ]. FLT3 ligand was mainly produced by bone marrow mesenchymal cells, the quantity of NK cells in mice lacking Flt3 ligand (Flt3L−/−) were obviously reduced [29]. FLT3 ligand coordinated with IL-15 could increase the ratio of NK cells derived from CD34+hematopoietic stem cell (HSC) notably than by using IL-15 alone, and the expression of both IL-2/IL-15 receptors on CD34+ HSC [30]. Cancer patients after auto-HSCT treated with subcutaneous injection of FLT3 ligand could significantly increase the number of dendritic cells and NK cells in vivo [31]. For these reasons, we tried to add SCF (protocol of group A) or SCF plus FLT3 ligand (protocol of group B) combined with IL-2, IL-7 and IL-15(protocol of group C) for 21 days of CB-CIK/NK cells expansion. Our study showed that, compared to group C, CD3+CD56+ CIK cells in group A was significantly decreased from an average of 24.03% to 19.84%, whereas that in group B was increased from an average of 24.03% to 26.02%. Solacingly, the proportion of CD3−CD56+ NK cells in group A and B group were both significantly increased from an average of 21.30% to 28.60% and 29.16%, respectively. Protocol in group B (five cytokines combined) seemed can also achieve the optimal effect on CB-CIK/NK cells proliferation compared to other groups, the expansion of CIK cells was about 800-fold (up to 1313-fold), and that of NK cells was about 36-fold in absolute numbers. These results showed that combination of IL-2, IL-7 and IL-15 with SCF alone might reduce CD3+CD56+ CIK cells yielding rate, but had some synergistic action on CD3−CD56+ NK expansion, nevertheless, CD3+CD56+ CIK cells and CD3−CD56+ NK cells could be both effectively expanded in the presence of IL-2, IL-7 and IL-15 combined with SCF/FLT3 ligand. After 21 days of cultivation, the proportion of CIK and NK cells in group A was more than 48%, while that in group B was more than 55%, indicating that combination of SCF and FLT3 ligand based on usage of IL-2, IL-7 and IL-15 were helpful for simultaneous expanding CD3+CD56+ CIK cells and CD3−CD56+ NK cells derived from cord blood. Our research result might be an important experimental evidence for clinical adoptive immunotherapy.

A previous report from Lu [13] by FACS sorting the cells then let them under the “classical” CIK cells expansion protocol showed that CD4+CD8− T cells did not develop expression of CD56,whereas the majority of cells that developed expression of CD56 originated from CD4−CD8+ cells in peripheral blood. Several studies by using the “classical” CIK cells expansion protocol illustrated that a considerable number of CD3+CD4+ and CD3+CD8+ T cells could be detected inside the culture system, were about 45% and 47%, respectively [1], another report of the amount of CD3+CD8+ T cells mixed in CIK cells “classical” expanding system could even be up to 67% [32]. For the percentage of CD3+CD8+T-cells in graft could have some relationship with GVHD [15, 16]. In our study, under the modified CIK/NK cells protocols, the proportions of CD8+CD3+ T cells on day 21 and day 14 appeared to have distinct differences, at harvest on day 21, CD8+CD3+ T cells only occupied about 10 to 20 percentage of the harvested effector cells, especially in group B and C. Our data show that on day 14 the total percentage of CIK cells, NK cells, CD4+ and CD8+ T cells in some groups were above 100%, this may be due to some CIK cells that also co-expressed CD8 markers overlapping with CD8+ T cells so the realistic amount CD8+ T cells in the culture system were less than the above data. Sometimes the lower amounts of CD8+CD3+ T cells mixed in the immune effectors would be more desirable in clinical adoptive immunotherapy, our modified protocol might be an alternative choice for CIK/NK cells expansion.

Finally, we compared the cytotoxic effect of various CB-CIK/NK cells expanded using the various protocols, the results indicated that the CIK/NK cells produced by the new protocols also possessed the killing activities against K562 cell line effectively, but the cytotoxicity did not parallel to the expansion capacity under different protocols. Previous studies showed that IL-2, IL-7 and IL-15 played an important role in maintaining the cytotoxic activity of killer cells [33, 34], and from Braun et al. [35], it has been confirmed that culture system containing SCF can enhance killing activity of lymphokine-activated killer cells against acute myeloid leukemia cell. In this study, we found that cytotoxicity of CIK/NK cells in group A, by adding SCF to the culture condition, was higher than that of group C, but if then added FLT3 ligand, such as under the protocol of group B, though the proliferate activity of CIK/NK cells in group B was improved, the cytotoxicity was weakened in some cases, it might be due to FLT3 ligand could impair the killing activity of NK cells [36].

We explore the efficacy of various combinations of stem cell factor, FLT3 ligand, interleukin (IL)-2, IL-7 and IL-15 on the induction and expansion of cord blood derived cytokine-induced killer cells. There were three groups, group A (SCF combined with IL-2, IL-7 and IL-15), group B (SCF, FLT3 ligand combined with IL-2, IL-7 and IL-15) and group C (IL-2, IL-7 and IL-15, control group). Proliferation rates of CD3⁺CD56⁺ CIK cells and CD3⁻CD56⁺ NK cells were highest in group B, expansion of CIK cells increased 796.1±278.5 fold, and that of NK cells increased 36.6+−3.5 fold. All expanded CB-CIK/NK cells showed cytotoxic activity against the K562 cell line. Interestingly, the cytotoxicity of group A was highest and significantly higher than that of other groups.

In our study, we noticed that combinations with more cytokines may help to produce more CIK and NK cells, but also may be accompanied by increasing numbers of CD8+ T cells in the cultivation system. Cultivation protocol of group B with both additional SCF and FLT3 ligand to IL-2, IL-7 and IL-15, might be optimal because it had higher capacity of expanding both CIK and NK cells with relatively less CD8+T cells production. Apart from increasing the killing activity of the CIK/NK cells against the K562 cell line, the protocol with only SCF plus IL-2, IL-7 and IL-15 (group A) showed no obvious advantage, rather fewer numbers of CIK cells and more unwanted CD8+ T cells in culture system compared to that of the basic IL-2, IL-7 and IL-15 protocol (group C). It is concluded that in the various combination of SCF, FLT3 ligand, IL-2,IL-7 and IL-15, we could develop modified protocols for cord blood derived CD3+CD56+ CIK and CD3−CD56+ NK cells expanding synchronously and effectively, and in containing less CD3+CD8+ T cells. This might provide an alternative choice for CIK/NK cell expansion in clinical applications.

TABLE 1
Phenotype of CB-CIK/NK cells in groups A, B and C on
days 14 and 21 of culture ( x̅ ± S, n = 9)

| | CD3+CD56+ CIK | | CD3−CD56+ NK | |
|---|---|---|---|---|
| | cells (%) | | cells (%) | |
| Group | d14 | d21 | d14 | d21 |
| A | 19.1 ± 2.1 | 19.8 ± 2.1★ | 26.2 ± 2.9 | 28.6 ± 1.5 |
| B | 21.3 ± 2.5 | 26.2 ± 4.1⋆ | 26.5 ± 1.4 | 29.2 ± 2.5 |
| C | 18.4 ± 3.3 | 24.0 ± 5.0⋆ | 28.7 ± 3.9 | 21.3 ± 2.0⋆★ |

⋆compared with that of d14 in the same group, p < 0.01
★compared with that of other groups on d21, p < 0.01

Groups A, B and C were generated as outlined in the “Materials and methods” section. Cells were stained with mAbs against CD3 conjugated to PerCP and CD56 conjugated to PE on days 14 and 21, and were analyzed by FACS. Mouse IgG1-PerCP and IgG2b-PE were used as negative controls.

TABLE 2
Expanding multiples of CB-CIK/NK cells in groups A, B and C on
days 14 and 21 of culture ( x̅ ± S, n = 9)

| | CD3+CD56+ CIK cells | | CD3−CD56+ NK cells | |
|---|---|---|---|---|
| Group | d14 | d21 | d14 | d21 |
| A | 447.9 ± 162.6 | 559.1 ± 174.0 | 33.3 ± 3.1 | 35.0 ± 7.9 |
| B | 563.8 ± 203.2^{□} | 796.1 ± 278.5⋆★ | 36.5 ± 5.9 | 36.6 ± 3.5 |
| C | 319.7 ± 116.2 | 575.8 ± 221.7⋆ | 37.2 ± 3.5 | 21.3 ± 4.8★ |

⋆compared with that of d14 in the same group, p < 0.01
□compared with that of group C on d14, p < 0.05
★compared with that of other groups on d21, p < 0.05

Groups A, B and C were generated as outlined in the “Materials and methods” section. Absolute numbers of CB-CIK/NK cells at different time points were determined by cell numbers calculated with a hemocytometer and the phenotype analyzed by flow cytometry.

TABLE 3
Phenotype of CD4+ and CD8+ T cells in groups A, B and C on
days 14 and 21 of culture ( x̅ ± S, n = 9)

| | CD3+CD4+ T | | CD3+CD8+ T | |
|---|---|---|---|---|
| | cells (%) | | cells (%) | |
| Group | d14 | d21 | d14 | d21 |
| A | 20.5 ± 4.8 | 17.4 ± 4.4 | 40.8 ± 5.2^{□} | 24.4 ± 5.2⋆★ |
| B | 23.2 ± 2.4 | 23.1 ± 2.6 | 48.3 ± 5.9 | 13.9 ± 2.8⋆ |
| C | 22.8 ± 5.3 | 22.4 ± 0.7 | 46.0 ± 9.2 | 10.4 ± 1.0⋆ |

⋆compared with that of d14 in the same group, p < 0.01
□compared with that of other groups on d14, p < 0.05
★compared with that of other groups on d21, p < 0.01

Groups A, B and C were generated as outlined in the “Materials and methods” section. CD4+ and CD8+ T cells were stained with mAbs against CD3 conjugated to PerCP, CD4 and CD8 conjugated to FITC, respectively, on days 14 and 21, and were analyzed by FACS. Mouse IgG1-PerCP and IgG1-FITC were used as negative controls.

### FIGURE LEGENDS

FIG. 1: Phenotype of CIK (FIG. 1A), NK (FIG. 1B) and T cells (FIG. 1C) on days 0, 14 and 21 of culture. Representative results from three experiments are shown.

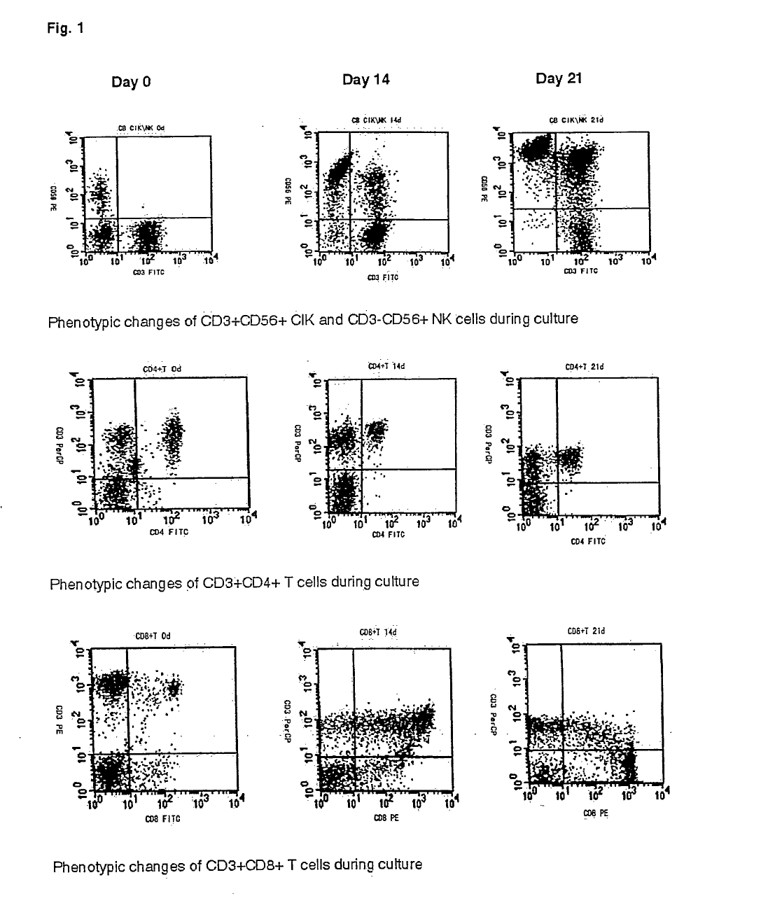

FIG. 2: Cytotoxicity of CIK/NK cells in groups A, B and C against the K562 cell line. CB-CIK/NK cells from protocols A, B and C were then used as effector cells in a cytotoxicity XTT assay against the K562 cell line at E:T ratio of 10:1 and 20:1. Representative results from nine experiments are shown. Results are expressed as the mean values of percent killing activities±SD. Cytotoxicity of CB-CIK/NK cells of groups A, B and C against the K562 cell line at E:T ratios of 10:1 were 55.33%±5.20%, 41.94%±4.18% and 45.68%±5.66%, respectively; and that at E:T ratios of 10:1 were 64.55%±5.74%,52.25%±5.10% and 54.57%±4.51%, respectively. Both killing activities of group A against K562 cell line at E:T ratios of 10:1 and 20:1 were significantly higher than that of group B and C and there was no difference between the cytotoxicities of group B and C.

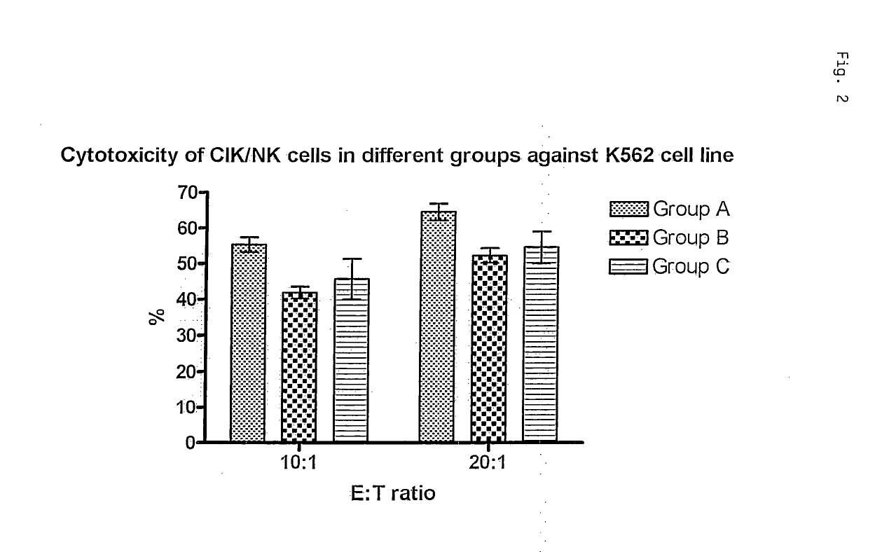

### REFERENCES

1. I. G. Schmidt-Wolf, P. Lefterova, B. A. Mehta, et al, Phenotypic characterization and identification of effector cells involved in tumor cell recognition of cytokine-induced killer cells. Exp Hematol. 21(13) (1993) 1673-1679.
2. S. Nagaraj, C. Ziske, I. G. Schmidt-Wolf, Human cytokine-induced killer cells have enhanced in vitro cytolytic activity via non-viral interleukin-2 gene transfer Genet Vaccines Ther. 25; 2(1) (2004) 12.
3. M. Introna, G. Borleri, E. Conti, et al, Repeated infusions of donor-derived cytokine-induced killer cells in patients relapsing after allogeneic stem cell transplantation: a phase I study. Hematol. 92(7) (2007) 952-959.
4. J. W. Shiver, S. Lishan, P. A. Henkart, et al. Cytotoxicity with target DNA break down by rat basophilic leukemia cells expressing both cytolysin and granzyme A. Cell. 71(2) (1992) 315-322.
C. Hoyle, C. D. Bangs, R. S. Negrin, et al, Expansion of Philadelphia chromosome-negative CD3(+)CD56(+) cytotoxic cells from chronic myeloid leukemia patients: in vitro and in vivo efficacy in severe combined immunodeficiency disease mice. Blood. 92(9) (1998) 3318-3327.
6. T. Leemhuis, S. Wells, C. Scheffold, et al, A phase I trial of autologous cytokine-induced killer cells for the treatment of relapsed Hodgkin disease and non-Hodgkin lymphoma. Biol Blood Marrow Transplant. 11(3) (2005) 181-187.
7. C. Scheffold, K. Brandt, I. G. Schmidt Wolf, et,al. Potential of autologus immunolgic effector cell for bone marrow purging in patients with chronic myeloid leukemia. Bone Marrow Transplant. 15(1) (1995) 33-39.
8. X. Shi-Xia, T. Xian-Hua, T. Xiang-Feng, Unrelated umbilical cord blood transplantation and unrelated bone marrow transplantation in children with hematological disease: A meta-analysis. Pediatr Transplant. 13(3) (2009) 278-284.
9. T. Wu, D. P. Lu, Blood and marrow transplantation in the People's Republic of China. Bone Marrow Transplant 42 Suppl 1 (2008) 73-75.
10. F. Locatelli, G. Giorgiani, A. Di-Cesare-Merlone, et, al, The changing role of stem cell transplantation in childhood. Bone Marrow Transplant. 41. Suppl 2 (2008) 3-7.
11. E. Gluckman, V. Rocha, Indications and results of cord blood transplant in children with leukemia. Bone Marrow Transplant. 41. Suppl 2 (2008) 80-82.
12. D T. Harris, In vitro and in vivo assessment of the graft-versus-leukemia activity of cord blood. Bone Marrow Transplant 15(1) (1995) 17-23.
13. P. H. Lu, R. S. Negrin, A novel population of expanded human CD3+CD56+ cells derived from T cells with potent in vivo antitumor activity in mice with severe combined immunodeficiency. J Immunol. 153(4) (1994) 1687-1696.
14. J. P. YU, X. B. Ren, P. Zhang, et al, Large-capacity expanded in vitro and biological characteristics assay of cytokine induced killer cells in malignant solid tumors patients. Chinese Journal of Cancer Biotherapy 8(3) (2001) 215-216.
15. K. Xu, C. Li, X. Pan, B. Du, et al, Study of relieving graft-versus-host disease by blocking CD137-CD137 ligand costimulatory pathway in vitro. Int J Hematol. 86(1) (2007) 84-90.
16. I. W. Abrahamsen, S. Somme, D. Heldal, et al, Immune reconstitution after allogeneic stem cell transplantation: the impact of stem cell source and graft-versus-host disease. Haematologica. 90(1) (2005) 86-93.
17. T. Meshulam, S. M. Levitz, L. Christin, et al, A simplified new assay for assessment of fungal cell damage with the tetrazolium dye, (2,3)-bis-(2-methoxy-4-nitro-5-sulphenyl)-(2H)-tetrazolium-5-carboxanilide (XTT). J Infect Dis. 172(4) (1995) 1153-1156.
18. B. A. Mehta, I. G. Schmidt-Wolf, I. L. Weissman, et al, Two pathways of exocytosis of cytoplasmic granule contents and target cell killing by cytokine-induced CD3+ CD56+ killer cells. Blood. 86 (1995) 3493-3499.
19. M. Introna, M. Franceschetti, A. Ciocca, et al, Rapid and massive expansion of cord blood-derived cytokine-induced killer cells: an innovative proposal for the treatment of leukemia relapse after cord blood transplantation. Bone Marrow Transplant. 38(9) (2006) 621-627.
20. N. Musha, Y. Yoshida, S. Sugahara, et al, Expansion of CD56+ NK T and gamma delta T cells from cord blood of human neonates. Clin Exp Immunol. 113(2) (1998) 220-228.
21. L. L. Lanier, A. M. Le, C. I. Civin, et al, The relationship of CD16 (Leu-11) and Leu-19 (NKH-1) antigen expression on human peripheral blood NK cells and cytotoxic T lymphocytes._Immunol. 136 (12) (1986) 4480-4486.
22. I. G. Schmidt-Wolf, R. S. Negrin, H. P. Kiem et al, Use of a SCID mouse/human lymphoma model to evaluate cytokine-induced killer cells with potent antitumor cell activity. J Exp Med. 174(1) (1991) 139-149.
23. I. G. Schmidt-Wolf, P. Lefterova, V. Johnston, et al, Propagation of large numbers of T cells with natural killer cell markers. Br J Haematol. 87(3) (1994) 453-458.
24. H. Ren, H. W. Xu, Y. H. Song, et al, The proliferation of CIK cells and its cytotoxicity on tumor cells in vitro. Chinese Journal of Immunology. 14(6) (1998) 406-408.
25. Z. Z. Kang, H. B. Cai, L. Xu, et al, Study on influence of different cultural conditions on CIK cells purity. Immunological Journal. 17(9) (2001) 385-387.
26. M. Wendel, I. E. Galani I E, Suri-Payer E, et al. Natural killer cell accumulation in tumors is dependent on IFN-gamma and CXCR3 ligands. Cancer Res. 68(20) (2008) 8437-8445.
27. W. H. Hallett, E. Ames, M. Alvarez, et al, Combination therapy using IL-2 and anti-CD25 results in augmented natural killer cell-mediated antitumor responses. Biol Blood Marrow Transplant. 14(10) (2008) 1088-1099.
28. H. Umekage, S. Saito, H. Morikawa, Enhancement by stem cell factor of interleukin-2 (IL-2)-induced DNA synthesis in human decidual CD16-CD56 bright natural killer cells mediated by increased expression of the IL-2 receptor alpha chain. J Reprod Immunol. 40(1) (1998) 1-24.
29. H. J. McKenna, K. L. Stocking, R. E. Miller, et al, Mice lacking flt3 ligand have deficient hematopoiesis affecting hematopoietic progenitor cells, dendritic cells, and natural killer cells. Blood. 1; 95(11) (2000) 3489-3497.
30. H. Yu , T. A. Fehniger , P. Fuchshuber, et al, FLT3 ligand promotes the generation of a distinct CD34(+) human natural killer cell progenitor that responds to interleukin-15. Blood. 92(10) (1998) 3647-3657.
31. N. Matsumura, M. Mandai, J. Hamanishi, et al, Immunostimulatory effect of Fms-like tyrosine kinase 3 ligand on peripheral monocyte-derived dendritic cells and natural killer cells: utilization for ovarian cancer treatment. Oncol Rep. 19(2) (2008) 505-515.
32. S. Finke, B. Trojaneck, P. Lefterova, et al, Increase of proliferation rate and enhancement of antitumor cytotoxicity of expanded human CD3+ CD56+ immunologic effector cells by receptor-mediated transfection with the interleukin-7 gene. Gene Ther. 5(1) (1998) 31-39.
33. J. S. Miller, J. Tessmer-Tuck, et al, Endogenous IL-2 production by natural killer cells maintains cytotoxic and proliferative capacity following retroviral-mediated gene transfer [J]. Exp Hematol. 25(11) (1997)1140-1148.
34. M. C. Mingari, C. Vitale, C. Cantoni, et al, Interleukin-15-induced maturation of human natural killer cells from early thymic precursors: selective expression of CD94/NKG2-A as the only HLA class I-specific inhibitory receptor [J]. Eur J Immunol. 27(6) (1997) 1374-1380.
35. S. Braun, H. H. Gerhartz, H. M. Schmetzer. Lymphokine-activated killer (LAK) cells and cytokines synergize to kill clonal cells in acute myeloid leukemia (AML) in vitro. Haematologia. 30 (4) (2000) 271-288.
36. N. Favre-Felix, M. Martin, E. Maraskovsky, et al, Flt3 ligand lessens the growth of tumors obtained after colon cancer cell injection in rats but does not restore tumor-suppressed dendritic cell function. J Cancer. 86(6) (2000) 827-834.

1. Method for generating and/or expansion of CIK cells and/or NK cells, the method comprising culturing blood cells in the presence of a culture medium comprising interleukin 15 (IL-15) and interleukin 7 (IL-7).

2. Method according to claim 1, wherein the culture medium additionally comprises interleukin 2 (IL-2).

3. Method according to claim 1, wherein the culture medium additionally comprises stem cell factor (SCF).

4. Method according to claim 1, wherein the culture medium additionally comprises Fms-related tyrosine kinase 3 ligand (FLT3 ligand).

5. Method according to claim 1, wherein the culture medium comprises IL-15 in an amount of about 4 to about 400 ng/ml, in particular about 10 to about 100 ng/ml.

6. Method according to claim 1, wherein the culture medium comprises IL-7 in an amount of about 4 to about 400 ng/ml, in particular about 10 to about 100 ng/ml.

7. Method according to claim 2, wherein the culture medium comprises IL-2 in an amount of about 8 to 800 ng/ml, in particular about 20 to about 200 ng/ml.

8. Method according to claim 3, wherein the culture medium comprises SCF in an amount of about 4 to about 400 ng/ml, in particular about 10 to about 100 ng/ml.

9. Method according to claim 4, wherein the culture medium comprises FLT3 ligand in an amount of about 4 to about 400 ng/ml.

10. Method according to claim 1, wherein the culture medium comprises IL-15, IL-7 and IL-2; and SCF, or FLT3 ligand, or SCF and FLT3 ligand.

11. Method according to claim 1, wherein the blood cells are peripheral blood cells.

12. Method according to claim 1, wherein the blood cells are mammal cells.

13. Method according to claim 1, wherein the blood cells are human cells.

14. Method according to claim 1, wherein the blood cells are derived from cord blood.

15. Kit for the generation and/or expansion of CIK cells and/or NK cells, comprising IL-15, IL-7 and IL-2; and facultatively either SCF, or FLT3 ligand, or SCF and FLT3 ligand.

16. CIK cell and/or NK cell population obtained by a method according to claim 1.

17. Method according to claim 1, wherein the culture medium comprises FLT3 ligand in an amount of about 10 to about 100 ng/ml.

18. CIK cell and/or NK cell population obtained by a method according to claim 2.

19. CIK cell and/or NK cell population obtained by a method according to claim 3.

20. CIK cell and/or NK cell population obtained by a method according to claim 4.
